(19) United States
(12) Patent Application Publication
(10) Pub. No.: US 2005/0043967 A1
(43) Pub. Date: Feb. 24, 2005

(54) Systems and methods for documenting home care services

(76) Inventors: Joseph R. Robinson, San Rafael, CA (US); David J. Mayne, Eagan, MN (US); Travis A. Morrell, San Francisco, CA (US); Kevin M. Chase, White Bear Lake, MN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/643,885

(22) Filed: Aug. 20, 2003

(51) Int. Cl. G06F 17/60

(52) U.S. Cl. 705/2

(57) ABSTRACT

The invention is directed to systems and methods for documenting home care services for providing reimbursement in the situation where a claimant has a relationship with a servicing entity. In particular, the invention relates to the situation where a claimant is being cared for by a caregiver. The method may comprise a caregiver entering the home of a claimant; the caregiver interfacing with a data input portion, which is disposed in the home of the claimant, prior to providing a service to the claimant so as to input a first data set into the data input portion; the caregiver providing the service to the claimant; upon completion of the service, the caregiver again interfacing with the data input portion to identify completion of the service, the first data set constituting collected data; and transmitting the collected data to the servicing entity.

### BACKGROUND OF THE INVENTION

A portion of the disclosure of this patent document contains material that is subject to copyright protection. The copyright owner has no objection to the electronic reproduction by anyone of the patent document or the patent disclosure, as it appears in the Patent and Trademark Office patent files or records, but otherwise reserves all copyright rights whatsoever.

The invention relates to home care providers working in the home of an individual who is associated with a servicing entity, such as an insurance company, wherein the servicing entity provides reimbursement for the services provided by the home care provider.

There is often times a substantial desire for persons to stay in their own home even when either the person's mental or physical health is failing or impaired in some manner. In order to allow such persons to remain in their home, home care providers are often utilized. A home care provider may provide a wide variety of services, such as feeding, dispensing medical supplies, and/or performing household duties, for example. Oftentimes, such home care providers, i.e., “caregivers,” are employed on an informal basis. For example, the caregiver might be a neighbor down the street.

Further, the person being cared for is typically associated with a servicing entity, such as an insurance company. In a typical situation, the person receiving care pays varying fees to the servicing entity, or in some other manner, has a relationship with the servicing entity. This relationship results in the servicing entity absorbing at least a portion or all of the cost of the home care. Oftentimes, this is effected by the person, who is receiving care, paying the caregiver once the caregiver completes the services in the home. Thereafter, the person receiving care might submit various documentation to the servicing entity so as to seek reimbursement under a so called “plan of care” that is in place. Accordingly, either in this or some other manner, a servicing entity provides a reimbursement for the services provided by the caregiver, and of course requires documentation in order to provide the reimbursement.

Using known processes and techniques, a typical claims reimbursement process has many data entry steps. Many of these steps often relate to entering reimbursement request information into a claims system of the servicing entity. The reimbursement request information may have been submitted on paper by facsimile or mail, for example. A reimbursement analysts may spend about thirty percent of their time entering the data that is on the paper invoice. As can be appreciated, such data entry is time consuming as well as susceptible to human error.

The systems and methods in accordance with the various embodiments of the invention address the above problems, as well as other problems that are present in known techniques and processes.

### BRIEF DESCRIPTION OF THE INVENTION

The invention is directed to systems and methods for documenting home care services for providing reimbursement in the situation where a claimant has a relationship with a servicing entity. In particular, the invention relates to the situation where a claimant is being cared for by a caregiver. The method may comprise a caregiver entering the home of a claimant; the caregiver interfacing with a data input portion, which is disposed in the home of the claimant, prior to providing a service to the claimant so as to input a first data set into the data input portion; the caregiver providing the service to the claimant; upon completion of the service, the caregiver again interfacing with the data input portion to identify completion of the service, the first data set constituting collected data; and transmitting the collected data to the servicing entity.

### BRIEF DESCRIPTION OF THE DRAWINGS

The present invention can be more fully understood by reading the following detailed description together with the accompanying drawings, in which like reference indicators are used to designate like elements, and in which:

FIG. 1 is a block diagram showing a care service system in accordance with one embodiment of the invention;

FIG. 2 is a flowchart showing a process for documenting home care services in accordance with one embodiment of the invention;

FIG. 3 is a block diagram showing in further detail the data input portion of FIG. 1 in accordance with one embodiment of the invention;

FIG. 4 is a block diagram showing in further detail the data processing entity of FIG. 1 in accordance with one embodiment of the invention;

FIG. 5 is a block diagram showing in further detail the servicing entity of FIG. 1 in accordance with one embodiment of the invention;

FIG. 6 is a highlevel flowchart showing a process for documenting home care services in accordance with a further embodiment of the invention;

FIG. 7 is a flowchart showing in further detail the “enroll program participates” step of FIG. 6 in accordance with an embodiment of the invention;

FIG. 8 is a flowchart showing in further detail the “perform installation of the data input portion” step of FIG. 6 in accordance with an embodiment of the invention;

FIG. 9 is a flowchart showing in further detail the “record caregiver time and services” step of FIG. 6 in accordance with an embodiment of the invention;

FIG. 10 is a flowchart showing in further detail the “perform transfer of data to servicing entity” step of FIG. 6 in accordance with an embodiment of the invention;

FIG. 11 is a flowchart showing in further detail the “perform reimbursement to caregiver” step of FIG. 6 in accordance with an embodiment of the invention;

FIG. 12 is a diagram showing the data input portion of FIG. 1 in further detail in accordance with one embodiment of the invention;

FIG. 13 is a diagram showing an illustrative invoice in accordance with one embodiment of the invention; and

FIG. 14 is a diagram showing illustrative schema for outputting data from a data processing entity to a servicing entity in accordance with one embodiment of the invention.

### DETAILED DESCRIPTION OF THE INVENTION

Hereinafter, aspects of the systems and methods in accordance with various embodiments of the invention will be described. As used herein, any term in the singular may be interpreted to be in the plural, and alternatively, any term in the plural may be interpreted to be in the singular.

The systems and methods of the invention provide a digital approach for claimants of a servicing entity, i.e., customers of insurance companies and other entities providing reimbursement services, to transmit cost of services invoice information to such servicing entities. Illustratively, a claims process typically requires that claimants submit invoices of their care services on a regular basis to the insurance company with which they are associated. The insurance company will then reimburse the claimant for the approved amount. In the past, this process has typically included a manual process that requires the claimant and caregiver to complete paper invoices and mail or fax them to the insurance company for reimbursement.

The disclosed systems and methods provide for placing a data input portion into the claimant's home. Buttons on the data input portion allow information to be entered that, in the past, is usually recorded using a paper invoice. The caregiver or care givers will use the data input portion to enter a caregiver code, the date and time of service, type of service, and cost of service, for example. This information will then be transmitted digitally to a claims system of the insurance company or other similar entity, i.e., a servicing entity, to expedite the reimbursement payment to the claimant. This transfer of data may occur automatically. Accordingly, the reimbursement process for customers of insurance entities will be easier and allow for quicker turn-around on reimbursement payments. Further, the disclosed systems and methods will allow, in many cases, for people to live in their homes longer. Generally, people prefer to stay in their homes rather than moving into a facility. The innovation will foster an environment in their homes where customers of servicing entities can live independently and safely.

The systems and methods described herein may be leveraged in any servicing entity where payment or service information needs to be transferred from the home to the servicing entity, or entity acting on behalf of the servicing entity, to provide reimbursement. As used herein, a “servicing entity” means any insurance company or other entity providing reimbursement for services provided in the home of a claimant.

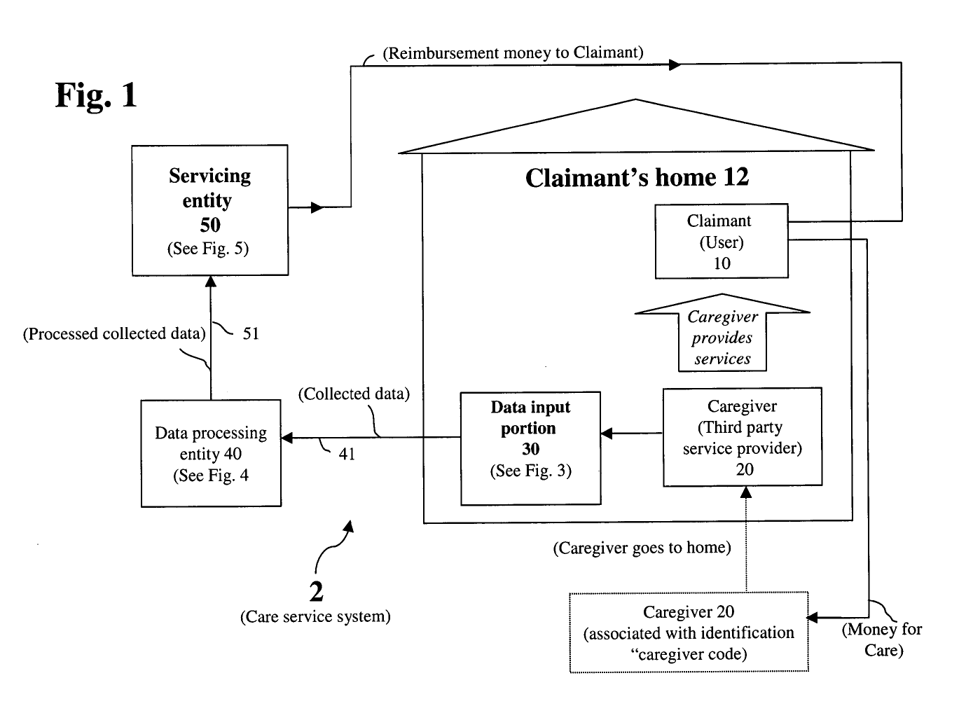

FIG. 1 is a block diagram showing a care service system in accordance with one embodiment of the invention. As shown in FIG. 1, the care service system 2 includes a claimant 10, i.e., a user of the care service system 2, and a caregiver 20. The caregiver 20 provides a variety of services to the claimant 10.

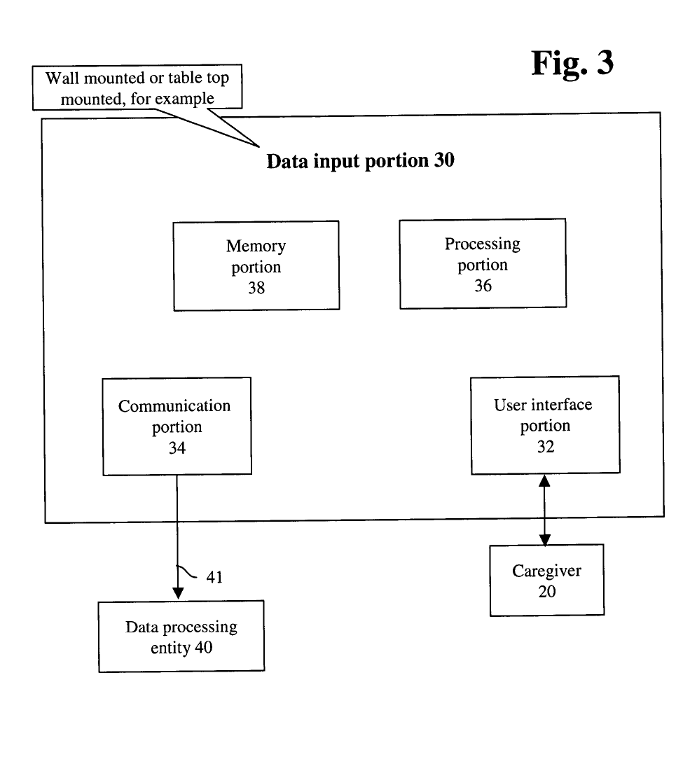

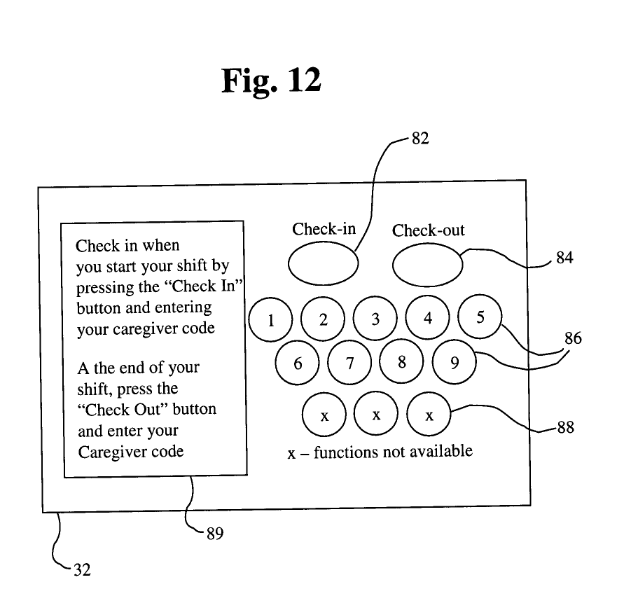

Accordingly, FIG. 1 shows a claimant home 12. In utilization of the systems and methods of the invention, a caregiver 20 will typically come to the home 12 of the claimant 10. While performing services in the claimant's home 12, the caregiver 20 interfaces with a data input portion 30 in conjunction with the performance of various services. Accordingly, the data input portion 30 collects a variety of information regarding who performed the services and the nature of services that were performed. Further details of the data input portion 30 are described below with reference to FIG. 3, as well as FIG. 12. The block diagram of FIG. 1 further includes a servicing entity 50. The servicing entity 50 receives data collected by the data input portion 30. Based on this data, the servicing entity 50 provides reimbursement, for example, to the caregiver 20.

As used herein, “home” means any place that a person resides or lives. Accordingly, a “home” as used herein includes an apartment, town home, single family house, duplex, condominium, or any other structure or collection of structures in which a person lives. In particular, “home” as used herein includes an assisted living facility, independent living facility, low-maintenance facility, or any other facility directed to housing elderly or impaired, for example, persons. Accordingly, a technical contribution and/or technical effect for the disclosed inventive technology is to provide systems and methods for documenting home care services, as set forth herein and in the Brief Description of the Invention, above.

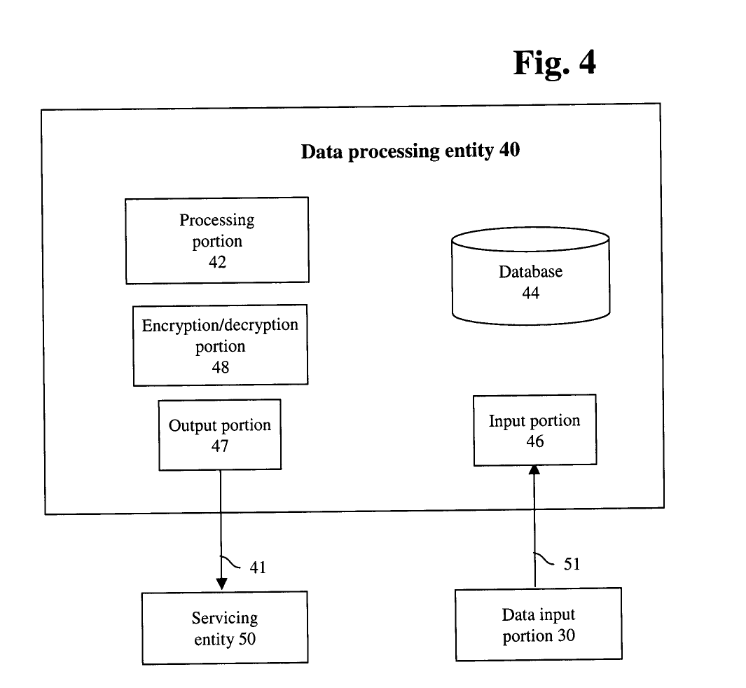

As shown in FIG. 1, the care service system 2 further includes a data processing entity 40. In accordance with one embodiment of the invention, the data processing entity 40 is utilized to provide a conduit between the data input portion 30 and the servicing entity 50. Further details of the data processing entity 40 are described below with reference to FIG. 4.

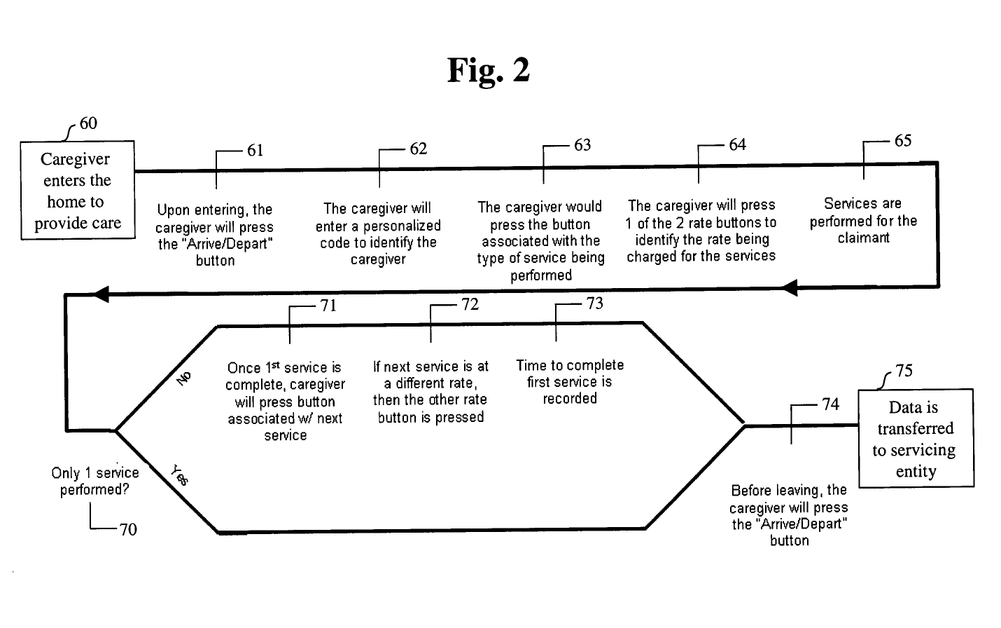

In further explanation, FIG. 2 is a flowchart showing a method in accordance with one embodiment of the invention. As shown, the process starts in step 60 in which the caregiver enters the home of the claimant, i.e., the user of the care service system, to provide care. In step 61, upon entering, the caregiver presses the “arrive/depart” button so as to identify an arrival time. Then, in step 62, the caregiver enters a personalized code to identify the caregiver. After step 62, the process passes to step 63.

In step 63, the caregiver presses a button associated with the type of service being performed. Then, the process passes to step 64. In step 64, in accordance with one embodiment of the invention, the caregiver will press one of two rate buttons. This action will identify the rate being charged for the services. It is, of course, appreciated that any number of rate buttons might be utilized as is necessary or desired. Accordingly, in steps 61-64, an arrival time, the identification of the caregiver, the type of service and rate information are obtained utilizing the data input portion 30 in accordance with one embodiment of the invention. Then the process passes to step 65.

In step 65, the caregiver provides any of a variety of services for the claimant in the home of the claimant. Such services might include feeding the claimant or dispensing medicine, for example. Then the process passes to step 70. Step 70 reflects the situation where more than one service is performed. If only one service is to be performed by the caregiver, then after step 70, the process passes to step 74. Alternatively, it may well be the situation that multiple services are performed by the caregiver in the claimant's home. Accordingly, the process passes from step 70 to step 71. That is, in step 71, once the first service is completed by the caregiver, the caregiver presses a button on the data input portion 30 associated with the next service. It is further noted that the next service might be at a different rate. Accordingly, in step 72, if the next service is at a different rate, then the caregiver may press the other rate button. After step 72, the process passes to step 73.

In step 73, the time to complete the first service is recorded. That is, in accordance with one embodiment of the invention, the caregiver does not expressly enter the completion time of the first service, but rather, the completion time of the first service is imputed from the progression into step 71 as shown in FIG. 2. After step 73, the process passes to step 74.

In step 74, the services of the caregiver are completed. Accordingly, the caregiver presses the “arrive/depart” button in the data input portion 30. Thereafter, the interaction between the data input portion 30 and the caregiver is terminated for that visit by the caregiver. After step 74, the process passes to step 75.

In step 75, the data now existing in the data input portion 30, i.e., the data relating to the services provided by the caregiver, is transferred or communicated to the servicing entity 50. This transfer of the data may be performed in a variety of ways, as is described in detail below. For example, the data might be transferred using a dial-up connection.

FIG. 3 is a block diagram showing the data input portion 30 of FIG. 1 in further detail. As shown in FIG. 3, the data input portion 30 interfaces with a caregiver 20 so as to input information regarding services that the caregiver 20 provides to the claimant 10. Further, the data input portion 30 outputs data to a data processing entity 40, i.e., data which the data input portion 30 has accumulated.

As shown in FIG. 3, the data input portion 30 includes a user interface portion 32. The user interface portion 32 may be an arrangement of buttons, for example, so as to interface with the caregiver 20 and obtain information regarding the services being provided by the caregiver 20. FIG. 12 shows further features of a user interface portion 32 in accordance with one embodiment of the invention.

Further, the data input portion 30 includes the communication portion 34. Once data is collected in the data input portion 30, the communication portion 34 outputs the collected data to the data processing entity 40 via a suitable interface 41. For example, the interface 41 might be in the form of a connection over the Internet or other network, or a dial-up connection, as noted above.

The data input portion 30 further includes additional components that perform the operations of the data input portion 30. That is, the data input portion 30 includes a processing portion 36. The processing portion 36 controls the various functions of the data input portion 30, including the input of information from a caregiver and the output of information to the data processing entity 40. Further, the data input portion 30 includes a memory portion 38. The memory portion 38 stores the necessary program code to effect operation of the data input portion 30. Further, the memory portion 38 stores the various data collected by the data input portion 30, i.e., prior to the data being output to the data processing entity 40.

FIG. 4 is a block diagram showing a data processing entity 40 in accordance with one embodiment of the invention. Accordingly, in accordance with this embodiment, an interim entity is utilized to effect communication of data from the data input portion 30 to the servicing entity 50. Accordingly, the data processing entity 40 assists in the collection and presentation of data to the servicing entity 50. As shown in FIG. 4, the data processing entity 40 includes a processing portion 42, a database 44, and an encryption/decryption portion 48. The processing portion 42 controls operations of the data processing entity 40. Further, the database 44 stores the various data manipulated in operation of the data processing entity 40.

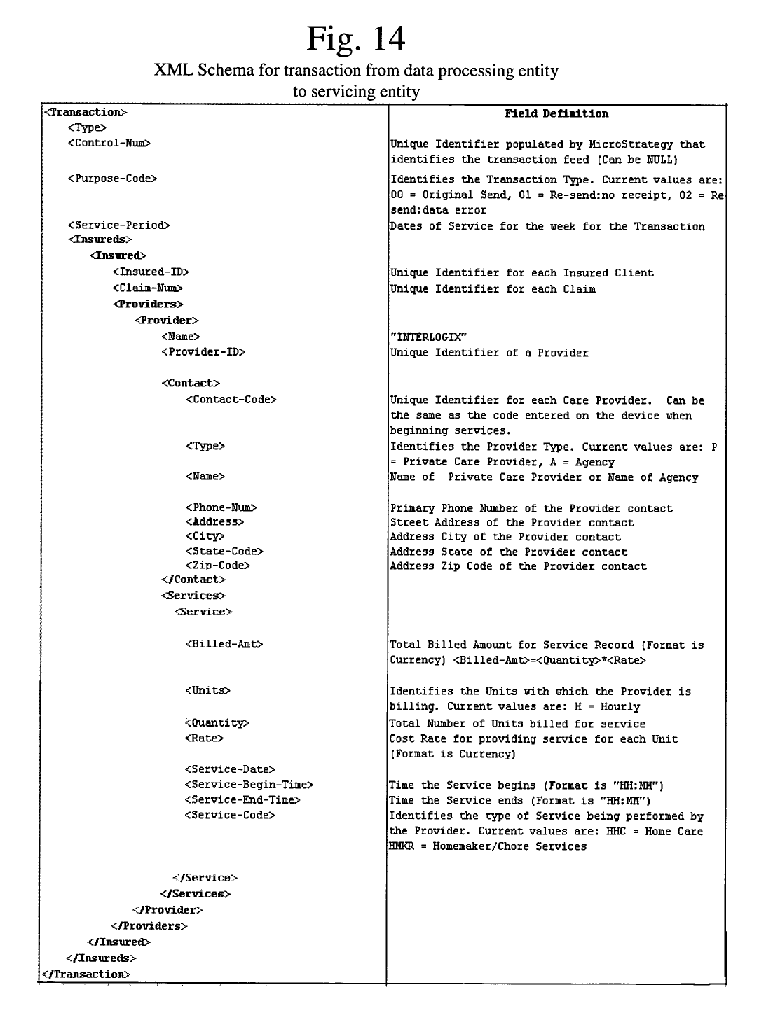

Further, the data processing entity 40 includes communication portions. That is, the data processing entity 40 includes an input portion 46 for interfacing with the data input portion 30. Further, the data processing entity 40 includes an output portion 47 for interfacing with the servicing entity 50. The interfaces 41 and 51 may be any suitable communication arrangement such as the Internet or suitable network. The data processing entity 40 may output data to the servicing entity 50 in a format as desired. FIG. 14 is a diagram showing schema that may be utilized to output data from the data processing entity 40 to the servicing entity 50.

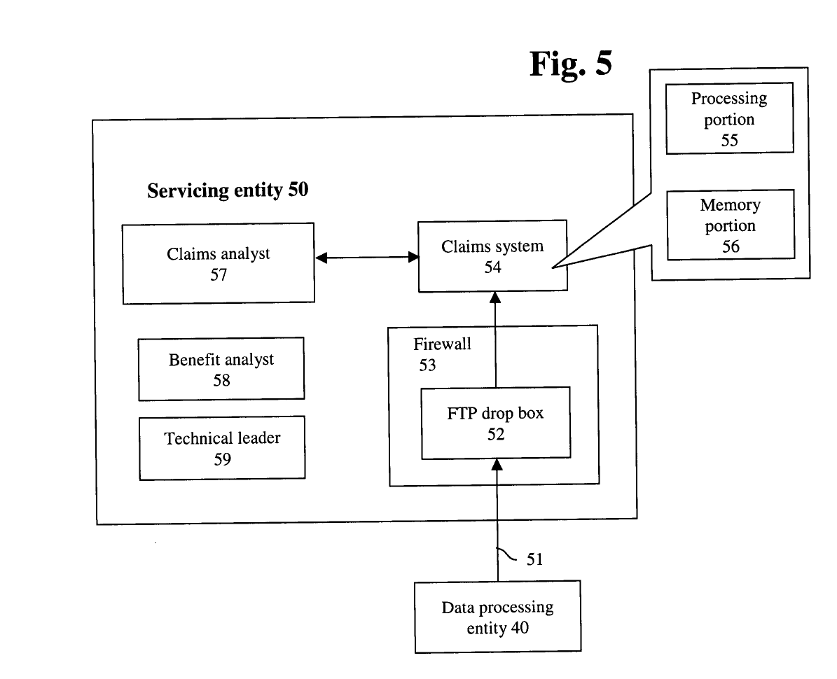

In further explanation of the invention, FIG. 5 is a flowchart showing the servicing entity 50 of FIG. 1 in further detail in accordance with one embodiment of the invention. As shown in FIG. 5, the servicing entity 50 inputs information from the data processing entity 40. In accordance with one embodiment of the invention, File Transfer Protocol (FTP) technology might be utilized. Accordingly, the servicing entity 50 includes an FTP drop box 52. Data input from the data processing entity 40 is initially input into the FTP drop box 52 in the servicing entity 50. Thereafter, the data passes through a firewall 53 into a claims system 54.

As shown in FIG. 5, the claims system 54 may include a processing portion 57 and a memory portion 58, which holds data obtained from the data processing entity 40. Once the data is collected and present in the claims system 54, the data may be reviewed and acted upon by a claims analyst 57. In accordance with one embodiment of the invention, the claims analyst 57 may be simply a person interfacing with the claims system 54 through a suitable interface. However, instead of a claims analyst 57 being a human person, it is appreciated that the claims analyst 57 might instead be in the form of a suitable processing machine. Further, the servicing entity 50 may include a benefit analyst 58. In accordance with one embodiment of the invention, the benefit analyst 58 is a person. Additionally, the servicing entity 50 may include a technical leader 59, which may also typically be a person. Further details of the servicing entity 50 including processing components and the persons effecting operation of the servicing entity 50 are described in detail below.

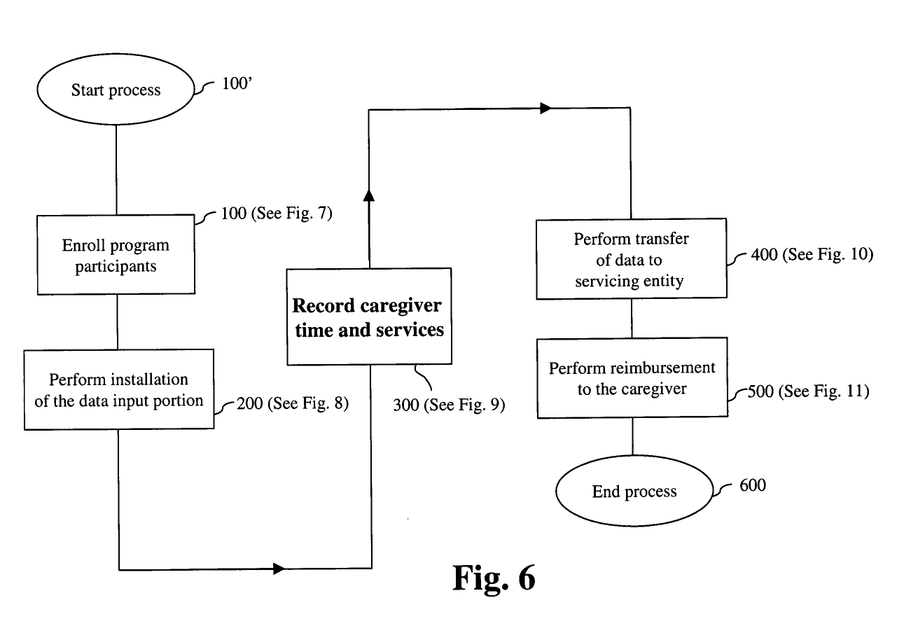

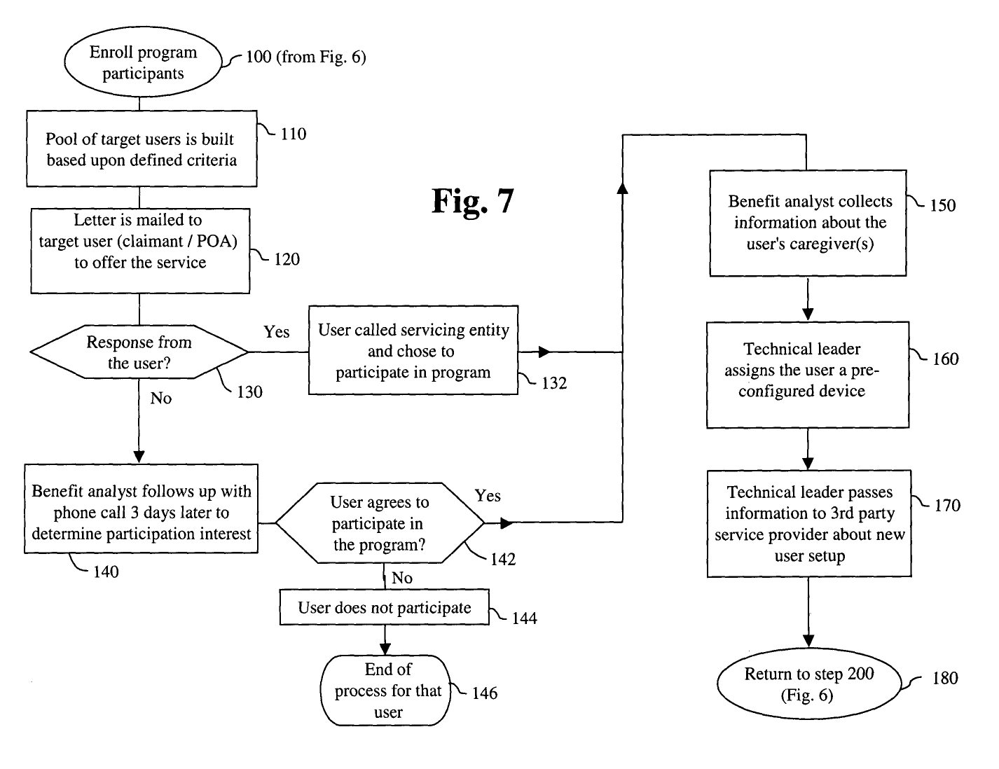

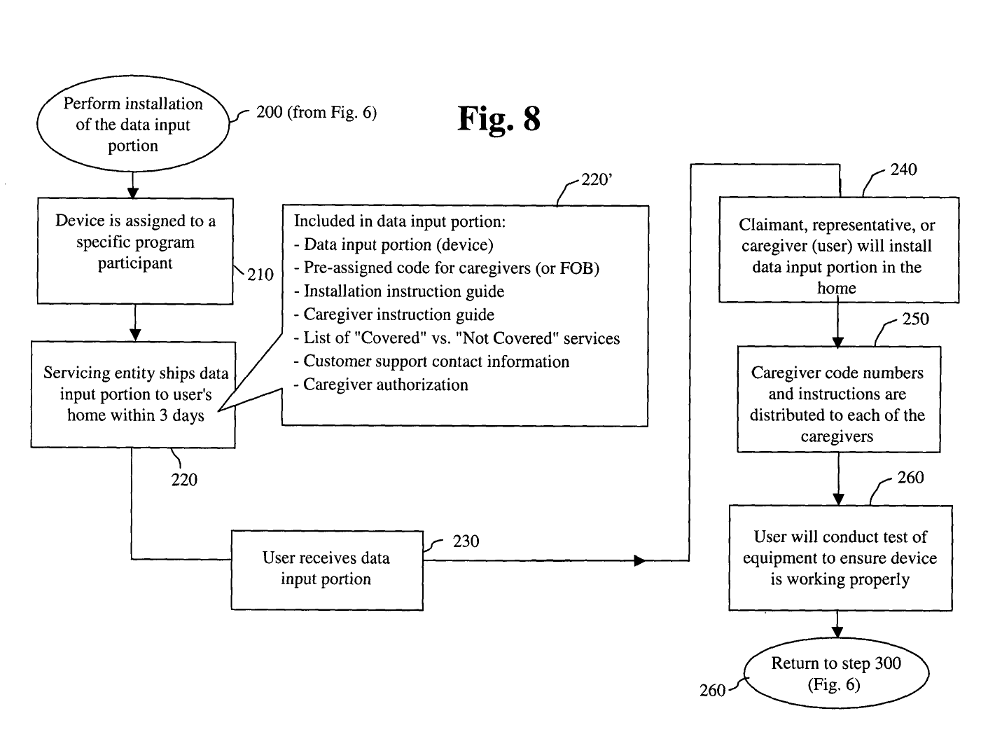

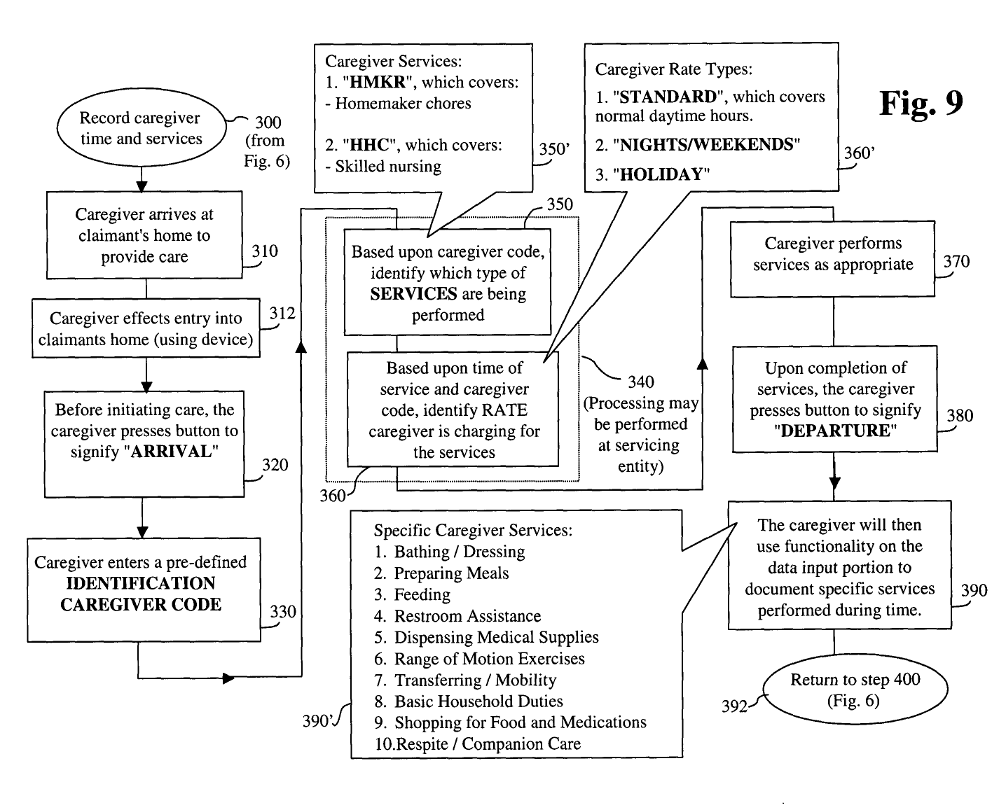

FIG. 6 is a high-level flowchart showing a process in accordance with a further embodiment of the invention. As shown in FIG. 6, the process is initiated in step 100′. Then, the process passes to step 100. In step 100, the process enrolls program participants. Further details of step 100 are discussed below with reference to FIG. 7. After step 100, the process passes to step 200. In step 200, installation of the data input portion is performed for new participants in the program. That is, a data input portion 30 is installed into the claimant's home 12. Further details of step 200 are described below with reference to FIG. 8. After step 200, the process passes to step 300. In step 300, the process records various information regarding the visit of the caregiver, including the time that was spent at the home by the caregiver as well as the services that were provided by the caregiver. Further details of step 300 are described below with reference to FIG. 9.

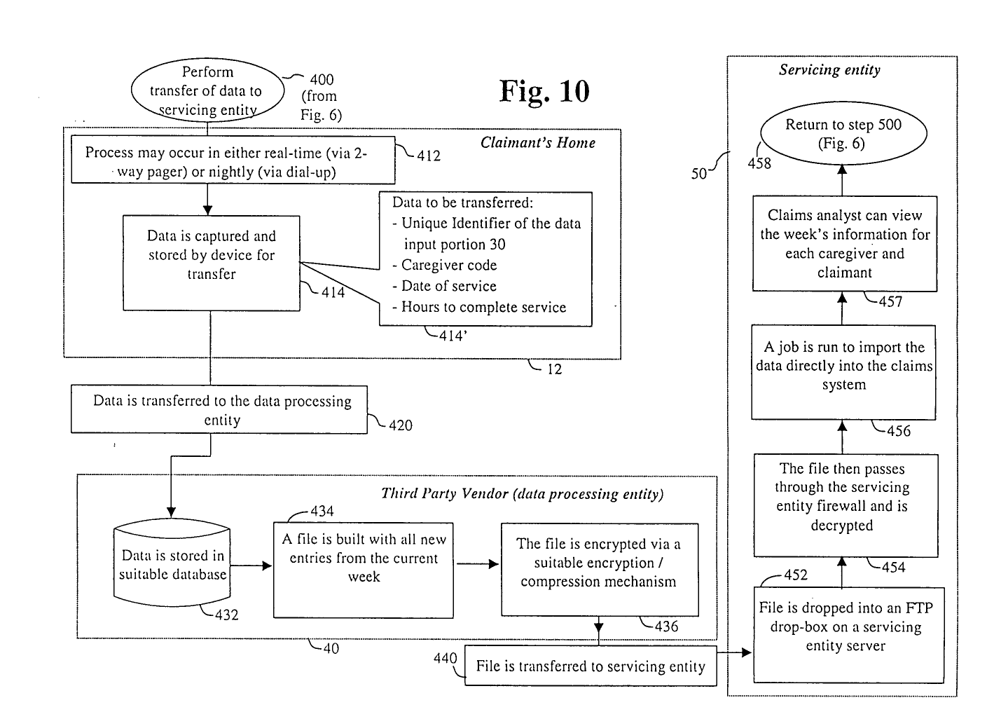

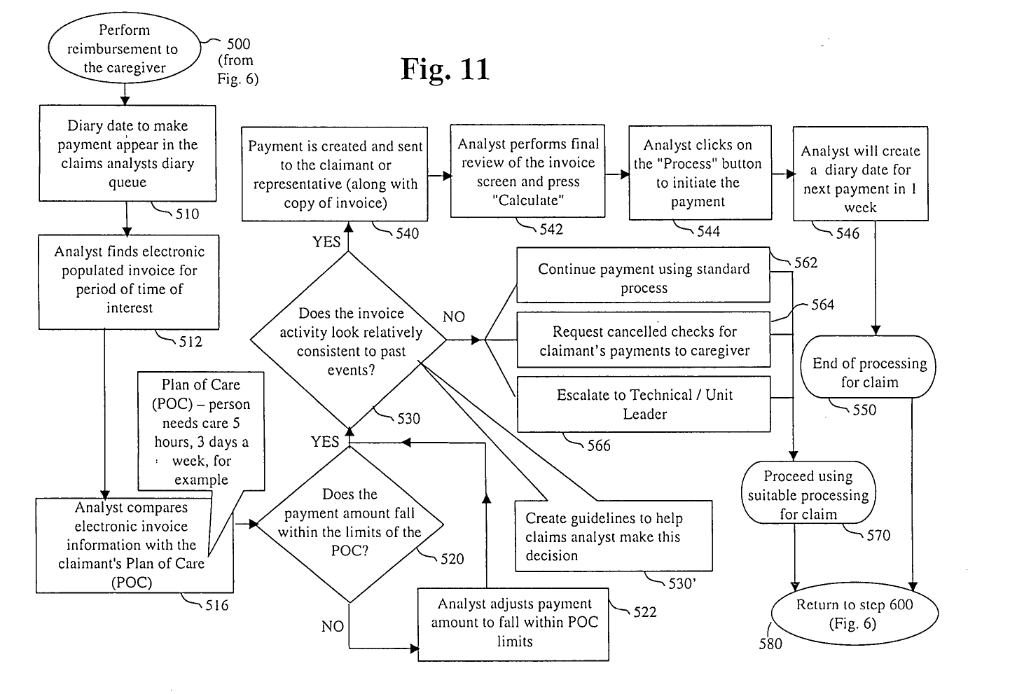

After step 300 of FIG. 6, the process passes to step 400. In step 400, the process performs a data transfer from the data input portion 30 in the claimants home 12 to the servicing entity 50. As noted above, in accordance with one embodiment of the invention, this transfer of data may be effected through a data processing entity 40. Further details of step 400 are described below with reference to FIG. 10. After step 400, the process passes to step 500. In step 500, reimbursement is performed to the caregiver. That is, upon receiving the information regarding the services provided by the caregiver, the servicing entity 50 effects an appropriate reimbursement to the caregiver. Further details of step 500 are described below with reference to FIG. 11. After step 500, the process passes to step 600. In step 600, the process ends.

FIG. 7 is a flowchart showing further details of the enroll program participants step 100 of FIG. 6 in accordance with one embodiment of the invention. As shown in FIG. 7, the process starts in step 100 and passes to step 110. In step 110, a pool of target users, i.e., claimants 10, is generated. The pool of target users may be built upon any of a variety of defined criteria or in any suitable manner. After step 110, the process passes to step 120. In step 120, an offer of the service is communicated to the target user. For example, a letter might be mailed to the target user providing information to the target user regarding the services and requesting the user to contact the servicing entity 50 to effect set up of the care service system.

After step 120, the process passes to step 130. In step 130, the process determines whether a response has been received from the user. This inquiry regarding any response from the targeted user might be performed in any desired period of time. For example, as shown in step 140, a benefit analyst might follow up with a phone call to the targeted user within three days to determine participation interest, i.e., in the case that a response has not been received from the targeted user. Assuming that contact and conversation was effected between a benefit analyst and the targeted user, the process passes from step 140 to step 142. In step 142, the process queries whether the targeted user agreed to participate in the program. If no, then the process passes to step 144, which reflects that the user did not choose to participate in the care service system. After step 144, the process passes to step 146 in which the process ends for that particular targeted user. Alternatively, as a result of discussions between the targeted user and the benefit analyst in the servicing entity 50, the targeted user might have indeed decided to participate in the care service system. Accordingly, the process passes from step 142 to step 150.

With further reference to step 130 of FIG. 7, it may be the situation that the targeted user did indeed call the servicing entity 50 to express their interest in participating in the care service system. That is, in step 132, the user chose to participate in the care service system. Accordingly, after either of steps 142 or 132, the process passes to step 150.

In step 150, the benefit analyst collects a variety of information about the user's caregiver or caregivers. Then, in step 160, the process may pass to a technical leader 59. The technical leader 59 assigns the now participating user 10 a preconfigured device, i.e., a preconfigured data input portion 30. Further, in step 170, the technical leader may pass information to the data processing entity 40 about the setup of the new user. After step 170, the process passes to step 180. In step 180, the process returns to step 200 of FIG. 6.

FIG. 8 is a flowchart showing in further detail the perform installation of the data input portion 30, step 200 of FIG. 6. As shown in FIG. 8, the process starts in step 200 and passes to step 210. In step 210, a data input portion 30 is assigned to a specific program participant. Then, in step 220, the servicing entity 50 ships the data input portion to the users home 12 within a suitable period of time. As shown in FIG. 8, a variety of information may be included with the shipping of the data input portion. That is, in addition to the device, further items and/or information which may be included include a pre-assigned code for caregivers or alternatively, a FOB to give to a caregiver, an installation instruction guide, a caregiver instruction guide, a list of covered versus. not covered services, customer support contact information and caregiver authorization, for example.

After step 220, the process passes to step 230. In step 230, FIG. 8 reflects the user receiving the data input portion. After step 230, the process passes to step 240. In step 240, a suitable person installs the data input portion in the home 12 of the claimant 10. For example, the installation might be of course performed by the claimant, some representative of the servicing entity 50, the caregiver or other suitable person. The data input portion 30 might be mounted on a wall or as a table top device, for example. After step 240, the process passes to step 250. In step 250, typically the claimant (user) distributes caregiver code numbers and instructions to each caregiver who is utilized by the claimant 10. Further, in step 260, as may be appreciated, the user may conduct suitable tests of the equipment to ensure that the device is working properly, as well as the communication channels between the device and the servicing entity 50. After step 260 of FIG. 8, the process passes to step 270. In step 270, the process returns to step 300 of the high-level flowchart of FIG. 6.

FIG. 9 is a flowchart showing further details of the record caregiver time and services step 300 of FIG. 6. As shown in FIG. 9, the process starts in step 300 and passes to step 310. In step 310, the caregiver 20 arrives at the claimant's home 12 to provide care. After step 310, the process passes to step 312. In step 312, the caregiver effects entry into the claimant's home, i.e., possibly using a suitable device or entry code. Then, in step 320, the caregiver presses a suitable button to signify the arrival of the caregiver. That is, the caregiver performs this action to signify arrival prior to beginning services. After step 320, the process passes to step 330. In step 330, in accordance with one embodiment of the invention, the caregiver 20 enters a predefined identification code. This identification code identifies the caregiver as a unique individual. After step 330, the process passes to step 350.

FIG. 12 is a diagram showing a user interface portion 32 of a data input portion 30 in accordance with one embodiment of the invention. The user interface portion 32 includes a check-in button 82 and a checkout button 84. Further, the user interface portion 32 includes numerical buttons 86. The numerical buttons 86 may be used by a caregiver to enter the caregiver's identification “caregiver code”. The buttons 82, 84 and 86 may be used to practice the process of FIG. 9 including steps 300 to 380. The user interface portion 32 of FIG. 12 also includes a “directions” panel 89, which provides directions for use of the data input portion 30.

Further, the user interface portion 32 includes buttons 88. The buttons 88 may be used to add functionality to the user interface portion 32. For example, functionality might be added so as to allow a caregiver to enter the additional information of step 390 of FIG. 9.

With further reference to FIG. 9, after step 330, the process passes to step 350. In step 350, the data input portion, based upon the caregiver code, prompts the caregiver 20 to identify which type of services are being performed. That is, for each caregiver, the data input portion 30 retrieves from memory the various types of services which might be performed by the particular caregiver and prompts the caregiver to identify which particular service is being performed. For example, as shown in step 350, caregiver services might be entered via a code HMKR or HHC, to identify homemaker chores and/or skilled nursing, respectively. Alternatively, the data input portion 30 might simply input the identification caregiver code, the type of service and/or the rate being assumed from the identification caregiver code either by the data input portion 30 or at the servicing entity 50, i.e., in the situation where the particular caregiver always performs the same type of service at the same rate.

After step 350 of FIG. 9, the process passes to step 360. In step 360, a rate is identified at which the caregiver services will be charged. For example, the rate might be based upon the time of the service as well as the particular caregiver, i.e., as indicated by the caregiver code. As shown in block 360′, caregiver rate types might include a standard rate for normal daytime hours, a night/weekend rate, as well as a holiday rate.

After step 360 of FIG. 9, the process passes to step 370. In step 370, the caregiver performs services as appropriate. Accordingly, it may be seen from FIG. 9 in accordance with one embodiment of the invention, that the data needed for the caregiver to enter into the data input portion 30, prior to beginning services, consists of only the caregiver code. Further, in step 380 in accordance with one embodiment of the invention, the caregiver simply presses the “departure button to signify completion of services.” Alternatively, the caregiver may again enter their caregiver code to signify completion of services, i.e., such that the data needed for the caregiver to enter after completing services also consists of only the caregiver code.

That is, in step 380, the caregiver completes the services. Accordingly, upon completion of the services, the caregiver presses a button to signify the “departure” of the caregiver, i.e., indicating the completion of services. Then, the process passes to step 390. In step 390, the caregiver will then use the functionality on the data input portion to document specific services performed during the time that the caregiver was at the claimant's home 12, in accordance with one embodiment of the invention. For example, specific caregiver services might include bathing or dressing, preparing meals, feeding, restroom assistance, dispensing medical supplies, assisting with motion exercises, transferring/mobility-related activities, basic household duties, shopping for food and medications, and/or respite/companion care, for example, as well as any of a variety of other caregiver services.

To explain further, step 350 of FIG. 9 may be used to provide the general nature of work that is provided. This general nature of work may either be assumed from the identification caregiver code or actually entered by the caregiver. In addition to step 350, step 390 of FIG. 9 may be used to collect more detailed information regarding the provided services.

As shown in FIG. 9, after step 390, the process passes to step 392. In step 392, the process returns to the high-level flowchart of FIG. 6 and particularly to step 400.

Accordingly, once the processing of FIG. 9 is completed, the caregiver has provided the requested services to the claimant and data representing those services and the person providing those services has been entered and is stored in the data input portion 30 in the claimant's home 12. Accordingly, after the processing of FIG. 9, the process passes to the processing of FIG. 10.

In further explanation of FIG. 9 in accordance with one embodiment of the invention, as discussed above, the caregiver enters the caregiver code in step 330, and based on the caregiver code, the servicing entity 50 later determines or imputes the particular type of service and the rate based on the caregiver code, i.e., as reflected in steps (350, 360) of FIG. 9. It is appreciated that it may be the situation that a particular caregiver has more than one rate and/or more than one type of service that is performed by the caregiver. In this situation, the process of FIG. 9 may simply flow from step 380 back to step 320. Upon returning to step 320, the caregiver would enter a new caregiver code, i.e., in other words, the caregiver would “re-log” into the data input portion 30.

Further, it is appreciated that step 390 of FIG. 9 is optional to the processing of FIG. 9 in accordance with one embodiment of the invention. That is, the additional layer of documentation of specific services that were performed by the caregiver may or may not be desired based on the particular situation. If the additional information is not desired, then the process may pass directly from step 380 to step 392.

FIG. 10 is a flowchart showing in further detail the “perform transfer of data to the servicing entity” step 400 of FIG. 6. As shown, FIG. 10 reflects processing in the claimant's home 12, processing by the data processing entity 40, as well as the arrival of data in and processing by the servicing entity 50.

As shown in FIG. 10, the process starts in step 400 and passes to step 412. Step 412 shows that the transfer process of data between the data input portion 30 and the data processing entity 40 may occur in either real time or via nightly dial-up, for example. If the processing is in real time, a 2-way pager might be utilized. Alternatively, a nightly dial-up once a day might be utilized to transmit the information from the data input portion 30 to the data processing entity 40. Alternatively, the Internet or other network might be used. It is appreciated that a wide variety of other technologies might be utilized to communicate the information from the data input portion 30 to the data processing entity 40. Further, step 414 further reflects that the data is captured by the data input portion 30 and stored by the device until transfer is effected.

In accordance with one embodiment of the invention, the data to be transferred may include a unique identifier for the particular data input portion 30, a caregiver code, the date of service, as well as the hours to complete the service, i.e., the arrival and departure dates of the caregiver. Other information such as type of service and rate may then be imputed based on the particular caregiver and/or the date of the service. As shown in FIG. 10, after step 414, the process passes to step 420.

In step 420, the data is transferred to the data processing entity. Then, FIG. 10 reflects processing by the data processing entity 40, i.e., which may be a suitable third-party vendor. That is, in step 432, the data is stored in a suitable database in the data processing entity 40. Then, in step 434, a file is built with all the new entries from a particular period of time, as desired. For example, the file might be built based on all the entries received by the data processing entity 40 within the prior week. The file may be built in a format agreed upon by the servicing entity 50 and the data processing entity 40. FIG. 14 illustrates schema that might be utilized in construction of a file by the data processing entity 40.

After step 434, the process passes to step 436. In step 436, the newly built file is encrypted utilizing a suitable encryption technique, for example. Further, as may be appreciated, suitable data compression techniques or mechanisms may also be utilized. After step 436, the process passes to step 440 in which the file is transferred to the servicing entity 50.

Thereafter, processing is effected by the servicing entity 50 as shown in FIG. 10. That is, in step 452, the file may be dropped into a FTP drop-box on the servicing entity server in accordance with one embodiment of the invention. Then, in step 454, the file passes through a servicing entity firewall and is decrypted. After step 454, the process passes to step 456.

In step 456, the data is imported into the claims system 54 in the servicing entity 50. For example, a suitable “job” might be run to import the data from the FTP drop-box into the claims system 54. Thereafter, the process passes to step 457. In step 457, a claims analyst interfaces with the claims system 54. That is, the claims analyst views the week's information for each caregiver and claimant. Then, the process passes to step 458. In step 458, the process returns to step 500 in the high-level flowchart of FIG. 6.

FIG. 11 is a flowchart showing the perform reimbursement to the caregiver, step 500 of FIG. 6. As shown in FIG. 11, the process starts in step 500 and passes to step 510. That is, FIG. 11 shows further details of the methodology used by the claims analyst 57 in interfacing with the claims system 54. In step 510 of FIG. 11, the claims analyst 57 diaries, i.e., calendars, a date to make the payment appear in the claims analyst diary queue. In other words, the claims analyst may be presented with a reimbursement request on a preliminary basis. After performing an initial review of the reimbursement request, the claims analyst 57 will then calendar a date to review and process the reimbursement request. After step 510, the process passes to step 512. In step 512, the analyst locates and opens an electronic populated invoice for a period of time of interest to the claims analyst. The invoices have now been populated with data. Then, the process passes to step 516.

In step 516, the claims analyst compares the invoice information with the claimant's Plan of Care (POC). Then the process passes to step 520. In step 520, the process determines, i.e., and more specifically the claims analyst determines, whether the payment amount falls within the limits of the plan of care. It may be the situation that the payment amount does not fall within the limits of the plan of care and accordingly, the process passes from step 520 to step 522. In step 522, the analyst adjusts a payment amount to fall within the plan of care limits. After step 522, the process proceeds to step 530. Alternatively, it may have been the situation in step 520, that the payment amount did indeed fall within the limits of the plan of care. Accordingly, the process passes directly from step 520 to step 530.

In step 530, the claims analyst, in accordance with one embodiment of the invention, determines whether the invoice activity appears relatively consistent to past events. As shown in the block 530′ of FIG. 11, various guidelines may be generated to help the claims analyst make this decision. If the invoice activity does indeed look relatively consistent with past events, then the process passes from step 530 to step 540. In step 540, the payment is created and sent to the claimant or representative, along with a copy of the invoice. After step 540, as shown in FIG. 11, the process passes to step 542. In step 542, the claims analyst performs a final review of the invoice screen and then presses “calculate.” That is, the claims analyst interfaces with the claims system to effect a final calculation of the amount due to the caregiver. As shown in FIG. 11, after step 542, the process passes to step 544. In step 544, the analyst clicks on the “process” button to initiate the payment to the caregiver. After step 544 of FIG. 11, the process passes to step 546. In step 546, the analyst creates a diary date for the next payment in one week

As shown in FIG. 11, after step 546, the process passes to step 570. Step 570 of FIG. 11 reflects the end of processing for that particular claim. That is, the data that was initially collected by the data input portion 30 from input by the caregiver 20 has resulted in a reimbursement to the caregiver. After step 570, the process passes to step 580 of FIG. 11. In step 580, the process returns to step 600 of FIG. 6.

With further reference to FIG. 11, it may be the situation in step 530 that the invoice activity does not look consistent with past events. As a result, the process passes from step 530 to any one of steps 582, 584 and/or 586. That is, in step 582, the processing of a particular request for reimbursement, for example, may be continued using a standard process. The standard process may likely reflect enhanced scrutiny of the request for reimbursement. Alternatively, or in addition to step 582, in step 584, further documentation might be required, such as canceled checks for the caregivers payments, i.e., canceled checks showing the transfer of monies from the claimant 10 to the caregiver 20 for past services. Further, the particular request for reimbursement might be escalated to a higher person at the servicing entity 50, i.e., higher than the claims analyst 57. For example, the request for reimbursement might be escalated to a technical or unit leader. After either of steps 582, 584 and/or 586, the process passes to step 570 as shown in FIG. 11. In step 570, the process proceeds utilizing suitable processing for the particular claim. Then, the process passes to step 580. In step 580, the process returns to step 600 of FIG. 6. As discussed above, in step 600 of FIG. 6, the process ends.

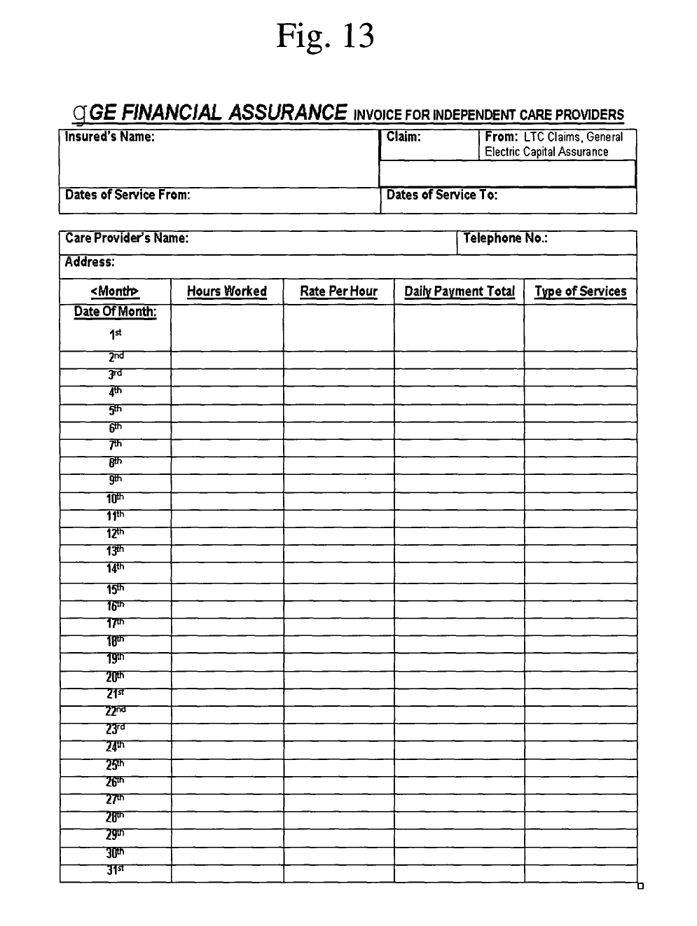

In the processing of FIG. 11, it is appreciated that it may be desired and/or necessary to provide documentation to either or both the claimant and the caregiver regarding data collected by the data input portion 30. This documentation might include the particular caregiver, the claimant, the date and time of service, as well as other information, for example. Illustratively, FIG. 13 shows an invoice for independent care providers. As shown, the invoice includes a variety of information including date, hours worked, rate, daily payment totals and type of service information for a particular insured. The invoice of FIG. 13 allows both the caregiver and the claimant, i.e., the insured, to be aware of exactly what was captured by the data input portion 30.

In accordance with one aspect of the invention, the disclosed method may be utilized to provide fraud detection, and in particular the invention may be used to reduce “soft fraud.” In particular, using known approaches, when a caregiver completes a paper invoice, the caregiver may often round up to the nearest hour. With the functionality of the data input portion 30, i.e., a device, the servicing entity 50 will know the exact point in time that care begins and ends, and as a result can reimburse exactly for those times. Further, the innovation also requires that caregivers be in the home to check-in and check-out. This is in contrast to the paper invoice process, where the caregiver simply had to record their hours once per month, which of course could be done anywhere at any time during the particular month.

In accordance with a further aspect of one embodiment of the invention, information obtained at the point of service by a caregiver is limited by the invention. To explain, it was discovered in developing the present invention that a large majority of the time, one caregiver always provides the same type of benefit. As a result, the method in one embodiment described herein, ties the benefit type into the unique caregiver identifier, thus eliminating the need for a caregiver to key both types of information into the device each time the caregiver performs a service.

In a similar manner, in development of the invention, it was noted that private care providers charge the same rate a large majority of the time. Accordingly, the method makes this assumption in accordance with one embodiment of the invention. In cases where the rate is different, system intelligence may be used to determine what the rate should be, i.e., standard, overtime, and/or holiday, for example.

Various embodiments of the systems and methods of the invention of the invention have been described above. The systems and methods of the invention may be implemented in conjunction with additional features. Such additional features might include, but are not limited to, activity tracking, notification of events, trend analysis and preventative medical treatment/advice, as well as other independent home care solutions.

To explain further, the systems and methods described herein may be expanded to include greater detail on the types of services being performed by the caregiver. As described above, high-level service codes (i.e. “HMKR—homemaker”) are captured to include all homemaker services. However, the data input portion might also include some sort of list functionality in order for the caregiver to specifically select the services he or she is performing, i.e., such as illustrated by step 390 of FIG. 9. This list may include, but is not limited to, bathing or dressing, preparing meals, feeding, restroom assistance, dispensing medical supplies, range of motion exercises, transferring or mobility related tasks, basic household duties, shopping for food and medications and/or respite/companion care. There might be either buttons labeled on the device, i.e., the data input portion, to capture this information or some type of display that would allow the caregiver to select the types of services being performed during the time period captured by the device.

In accordance with a further aspect of one embodiment of the invention, because caregiver information may change quite frequently, the innovation may be provided and have the capacity for easy, self-guided maintenance of this type of information, such as new caregiver added, caregiver removed, and/or caregiver data changes. The innovation may be provided with a web service and/or a telephone call-in system so that the caregiver or claimant can self-administer the maintenance of this type of information.

As noted herein, the systems and methods in accordance with some embodiments of the invention, may be combined with various functionality. For example, the care service system described above may be combined with a personal emergency response system. Such a service might include an alert mechanism, such as a wristband push button, that an individual wears on their person to use in case of a medical emergency. The capability to provide this functionality may be incorporated into the data input portion described above, i.e., the data input portion and the alert mechanism would be in communication. Upon the activation of the alert mechanism, the data input portion would connect to an operator who would determine a person's emergency needs. The alert mechanism might also be triggered to connect to a friend or relative.

Further, the systems and methods, in accordance with some embodiments of the invention, may be combined with a home security system. Such a home security system might provide for door monitoring, window monitoring, and/or secured access, for example.

Further, the systems and methods, in accordance with some embodiments of the invention, may be combined with activity monitoring. Activity monitoring might be performed through sensors or motion detectors. For example. Such devices might be placed in the home to better monitor and track a person's activities. Information from these sources can be used to trigger events, such as a phone call or e-mail if no activity takes place during a certain time period. This data can also be used to proactively monitor trends in a person's eating, sleeping, and other behaviors to diagnose preventative health treatments. Such information might be communicated to the data input portion, and then communicated onto the servicing entity, as such components are shown in FIG. 1.

Accordingly, the invention is intended to target informal home health care providers, such as a neighbor or friend. The invention makes the process very simple for such caregivers, which is beneficial since the primary skill set of such caregivers will usually be in the area of providing the care, rather than “tracking” the services provided.

As described above, data is collected by the data processing entity 40 and then output to the servicing entity 50 in a particular format, as agreed upon by the data processing entity 40 and the servicing entity 50. Any suitable third party might be used for the processing of data as performed by the data processing entity 40. For example, the entity MICRO STRATEGY might be utilized as the data processing entity 40.

As noted above, FIG. 14 shows illustrative schema that may be used in transferring data from the data processing entity 40 to the servicing entity 50. Further, set forth below, is an illustrative file that follows and is to be used in conjunction with the schema of FIG. 14. Accordingly, illustrative data is set forth below that follows the schema of FIG. 14. However, it is of course appreciated that the invention is not limited to the schema of FIG. 14 and the file content below. Rather, the schema and file content may be varied as should be appreciated by one of ordinary skill in the art.

```
- <Transaction>
    <Type>INTLGX</Type>
    <Control_Num />
    <Purpose_Code>00(Original)</Purpose_Code>
    <Service_Period>20030723-20030729</Service_Period>
  - <Insureds>
        - <Insured>
            <Insured_ID>363636363</Insured_ID>
            <Claim_Num>A7777777</Claim_Num>
          - <Providers>
            - <Provider>
                <Name>INTERLOGIX</Name>
                <Provider_ID>12345</Provider_ID>
              - <Contact>
                  <Contact_Code>1324</Contact_Code>
                  <Type>A</Type>
                  <Name>Bob's Home Care, Inc.</Name>
                  <Phone_Num>5551234567</Phone_Num>
                  <Address>1204 Inverness Way</Address>
                  <City>Mt. Hill</City>
                  <State_Code>MA</State_Code>
                  <Zip_Code>22222</Zip_Code>
                </Contact>
              - <Services>
                - <Service>
                    <Billed_Amt>120</Billed_Amt>
                    <Units>H</Units>
                    <Quantity>8</Quantity>
                    <Rate>15</Rate>
                    <Service_Date>20030724</Service_Date>
                    <Service_Begin_Time>12:00</Service_Begin_{—}
                    Time>
                  <Service_End_Time>20:00</Service_End_Time>
                  <Service_Code>HHC</Service_Code>
                </Service>
              </Services>
            </Provider>
          </Providers>
        </Insured>
      - <Insured>
          <Insured_ID>111111111</Insured_ID>
          <Claim_Num>A987654</Claim_Num>
        - <Providers>
          - <Provider>
              <Name>INTERLOGIX</Name>
              <Provider_ID>12345</Provider_ID>
            - <Contact>
                <Contact_Code>6985</Contact_Code>
                <Type>P</Type>
                <Name>Suzy Caregiver</Name>
                <Phone_Num>5559876543</Phone_Num>
                <Address>227 4th Street</Address>
                <City>Pleasantville</City>
                <State_Code>ME</State_Code>
                <Zip_Code>44444</Zip_Code>
              </Contact>
            - <Services>
              - <Service>
                  <Billed_Amt>120</Billed_Amt>
                  <Units>H</Units>
                  <Quantity>12</Quantity>
                  <Rate>10</Rate>
                  <Service_Date>20030725</Service_Date>
                    <Service_Begin_Time>8:00</Service_Begin_{—}
                    Time>
                  <Service_End_Time>20:00</Service_End_Time>
                  <Service_Code>HMKR</Service_Code>
                </Service>
              - <Service>
                  <Billed_Amt>80</Billed_Amt>
                  <Units>H</Units>
                  <Quantity>8</Quantity>
                  <Rate>10</Rate>
                  <Service_Date>20030726</Service_Date>
                    <Service_Begin_Time>12:00</Service_Begin_{—}
                    Time>
                  <Service_End_Time>20:00</Service_End_Time>
                  <Service_Code>HMKR</Service_Code>
                </Service>
                </Services>
              </Provider>
            - <Provider>
                <Name>INTERLOGIX</Name>
                <Provider_ID>12345</Provider_ID>
              - <Contact>
                  <Contact_Code>8954</Contact_Code>
                  <Type>P</Type>
                  <Name>Johnny Neighbor</Name>
                  <Phone_Num>5551112233</Phone_Num>
                  <Address>1008 Sacramento Street</Address>
                  <City>White Plains</City>
                  <State_Code>WA</State_Code>
                  <Zip_Code>99988</Zip_Code>
                </Contact>
              - <Services>
                - <Service>
                    <Billed_Amt>90</Billed_Amt>
                    <Units>H</Units>
                    <Quantity>6</Quantity>
                    <Rate>15</Rate>
                    <Service_Date>20030727</Service_Date>
                      <Service_Begin_Time>10:00</Service_{—}
                      Begin_Time>
                    <Service_End_Time>16:00</Service_End_{—}
                    Time>
              <Service_Code>HMKR</Service_Code>
            </Service>
          </Services>
        </Provider>
      </Providers>
    </Insured>
  </Insureds>
</Transaction>
```

Hereinafter, general aspects of possible implementation of the inventive technology will be described. Various embodiments of the inventive technology are described above. In particular, various steps of embodiments of the processes of the inventive technology are set forth. Further, various illustrative operating systems are set forth. It is appreciated that the systems of the invention or portions of the systems of the invention may be in the form of a “processing machine,” such as a general purpose computer, for example. As used herein, the term “processing machine” is to be understood to include at least one processor that uses at least one memory. The at least one memory stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as those tasks described above in the flowcharts. Such a set of instructions for performing a particular task may be characterized as a program, software program, or simply software.

As noted above, the processing machine executes the instructions that are stored in the memory or memories to process data. This processing of data may be in response to commands by a user or users of the processing machine, in response to previous processing, in response to a request by another processing machine and/or any other input, for example.

As noted above, the processing machine used to implement the invention may be a general purpose computer. However, the processing machine described above may also utilize any of a wide variety of other technologies including a special purpose computer, a computer system including a microcomputer, mini-computer or mainframe for example, a programmed microprocessor, a micro-controller, a peripheral integrated circuit element, a CSIC (Customer Specific Integrated Circuit) or ASIC (Application Specific Integrated Circuit) or other integrated circuit, a logic circuit, a digital signal processor, a programmable logic device such as a FPGA, PLD, PLA or PAL, or any other device or arrangement of devices that is capable of implementing the steps of the process of the invention.

It is appreciated that in order to practice the method of the invention as described above, it is not necessary that the processors and/or the memories of the processing machine be physically located in the same geographical place. That is, each of the processors and the memories used in the invention may be located in geographically distinct locations and connected so as to communicate in any suitable manner. Additionally, it is appreciated that each of the processor and/or the memory may be composed of different physical pieces of equipment. Accordingly, it is not necessary that a processor be one single piece of equipment in one location and that the memory be another single piece of equipment in another location. That is, it is contemplated that the processor may be two pieces of equipment in two different physical locations. The two distinct pieces of equipment may be connected in any suitable manner. Additionally, the memory may include two or more portions of memory in two or more physical locations.

To explain further, processing as described above is performed by various components and various memories. However, it is appreciated that the processing performed by two distinct components as described above may, in accordance with a further embodiment of the invention, be performed by a single component. Further, the processing performed by one distinct component as described above may be performed by two distinct components. In a similar manner, the memory storage performed by two distinct memory portions as described above may, in accordance with a further embodiment of the invention, be performed by a single memory portion. Further, the memory storage performed by one distinct memory portion as described above may be performed by two memory portions.

Further, various technologies may be used to provide communication between the various processors and/or memories, as well as to allow the processors and/or the memories of the invention to communicate with any other entity; i.e., so as to obtain further instructions or to access and use remote memory stores, for example. Such technologies used to provide such communication might include a network, the Internet, Intranet, Extranet, LAN, an Ethernet, or any client server system that provides communication, for example. Such communications technologies may use any suitable protocol such as TCP/IP, UDP, or OSI, for example.

As described above, a set of instructions is used in the processing of the invention. The set of instructions may be in the form of a program or software. The software may be in the form of system software or application software, for example. The software might also be in the form of a collection of separate programs, a program module within a larger program, or a portion of a program module, for example The software used might also include modular programming in the form of object oriented programming. The software tells the processing machine what to do with the data being processed.

Further, it is appreciated that the instructions or set of instructions used in the implementation and operation of the invention may be in a suitable form such that the processing machine may read the instructions. For example, the instructions that form a program may be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processing machine, i.e., to a particular type of computer, for example. The computer understands the machine language.

Any suitable programming language may be used in accordance with the various embodiments of the invention. illustratively, the programming language used may include assembly language, Ada, APL, Basic, C, C++, COBOL, dBase, Forth, Fortran, Java, Modula-2, Pascal, Prolog, REXX, Visual Basic, and/or JavaScript, for example. Further, it is not necessary that a single type of instructions or single programming language be utilized in conjunction with the operation of the system and method of the invention. Rather, any number of different programming languages may be utilized as is necessary or desirable.

Also, the instructions and/or data used in the practice of the invention may utilize any compression or encryption technique or algorithm, as may be desired. An encryption module might be used to encrypt data. Further, files or other data may be decrypted using a suitable decryption module, for example.

As described above, the invention may illustratively be embodied in the form of a processing machine, including a computer or computer system, for example, that includes at least one memory. It is to be appreciated that the set of instructions, i.e., the software for example, that enables the computer operating system to perform the operations described above may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in the invention may take on any of a variety of physical forms or transmissions, for example. Illustratively, the medium may be in the form of paper, paper transparencies, a compact disk, a DVD, an integrated circuit, a hard disk, a floppy disk, an optical disk, a magnetic tape, a RAM, a ROM, a PROM, a EPROM, a wire, a cable, a fiber, communications channel, a satellite transmissions or other remote transmission, as well as any other medium or source of data that may be read by the processors of the invention.

Further, the memory or memories used in the processing machine that implements the invention may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the system and method of the invention, a variety of “user interfaces” may be utilized to allow a user to interface with the processing machine or machines that are used to implement the invention. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, voice reader, voice recognizer, dialogue screen, menu box, list, checkbox, toggle switch, a pushbutton or any other device that allows a user to receive information regarding the operation of the processing machine as it processes a set of instructions and/or provide the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the system and method of the invention, it is not necessary that a human user actually interact with a user interface used by the processing machine of the invention. Rather, it is contemplated that the user interface of the invention might interact, i.e., convey and receive information, with another processing machine, rather than a human user. Accordingly, the other processing machine might be characterized as a user. Further, it is contemplated that a user interface utilized in the system and method of the invention may interact partially with another processing machine or processing machines, while also interacting partially with a human user.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

Accordingly, while the present invention has been described here in detail in relation to its exemplary embodiments, it is to be understood that this disclosure is only illustrative and exemplary of the present invention and is made to provide an enabling disclosure of the invention. Accordingly, the foregoing disclosure is not intended to be construed or to limit the present invention or otherwise to exclude any other such embodiments, adaptations, variations, modifications and equivalent arrangements.

1. A method for documenting home care services for providing reimbursement in the situation where a claimant has a relationship with a servicing entity, the claimant being cared for by a caregiver, the method comprising:
a caregiver entering the home of a claimant;
the caregiver interfacing with a data input portion, which is disposed in the home of the claimant, prior to providing a service to the claimant so as to input a first data set into the data input portion;
the caregiver providing the service to the claimant;
upon completion of the service, the caregiver again interfacing with the data input portion to identify completion of the service, the first data set constituting collected data; and
transmitting the collected data to the servicing entity.

2. The method of claim 1, wherein, upon completion of the service, the caregiver again interfacing with the data input portion to identify completion of the service and so as to input a second data set into the data input portion, the first data set and the second data set constituting the collected data.

3. The method of claim 2, wherein the second data set includes at least one of a caregiver code and service information.

4. The method of claim 1, wherein the first data set consists of a caregiver code.

5. The method of claim 1, wherein the first data set comprises at least one of a caregiver code, type of service information and hourly rate information.

6. The method of claim 1, wherein the transmitting the collected data to the servicing entity is performed using a data processing entity, and wherein:
the data processing entity inputting the collected data from the data input portion;
the data processing entity processing the collected data to generate processed collected data; and
the data processing entity outputting the processed collected data to the servicing entity.

7. The method of claim 1, wherein the transmitting the collected data to the servicing entity is performed in real time.

8. The method of claim 1, wherein the transmitting the collected data to the servicing entity is performed on a daily basis.

9. The method of claim 8, wherein the transmitting the collected data to the servicing entity is performed using a dial up connection.

10. The method of claim 1, wherein the first data set includes caregiver code, and further including, after transmitting the collected data to the servicing entity, the servicing entity determining the service type and the hourly rate based on the caregiver code.

11. The method of claim 1, further including the servicing entity determining a reimbursement amount based on the collected data, the reimbursement amount being the amount of monies to be forwarded from the servicing entity to the claimant so that the claimant may be reimbursed for payments to the caregiver.

12. The method of claim 11, further including the servicing entity preparing and sending an invoice to at least one of the caregiver and the claimant, the invoice documenting information obtained by the servicing entity from the data input portion and the collected data.

13. The method of claim 1, wherein the data input portion is disposed in the claimant's home for an extended period of time, so that the same data input portion is used by a plurality of caregivers.

14. The method of claim 13, further including mounting the data input portion on a wall in the claimant's home.

15. The method of claim 13, further including disposing the data input portion as a table top device in the claimant's home.

16. The method of claim 1, wherein the method is performed in sequential order.

17. The method of claim 1, wherein the first data set includes a caregiver code of the caregiver, and the method further including, after the caregiver has input second information indicating completion of the service;
the caregiver inputting a third data set, the third data set being entered prior to the caregiver beginning additional service for the claimant, and the third data set being associated with at least one of a different rate and type of service from the first data set.

18. The method of claim 1, wherein the caregiver is a neighbor of the claimant, living proximate to the claimant.

19. The method of claim 1, wherein the servicing entity is an insurance company.

20. A system for documenting home care services for providing reimbursement in the situation where a claimant has a relationship with a servicing entity, the claimant being cared for by a caregiver, the system comprising:
a data input portion disposed in the home of the claimant, a caregiver entering the home of a claimant and interfacing with the data input portion prior to providing a service to the claimant so as to input a first data set into the data input portion, and upon completion of the service, the caregiver again interfacing with the data input portion to identify completion of the service and so as to input a second data set into the data input portion, the first data set and the second data set constituting collected data; and
a servicing entity, the data input portion transmitting the collected data to the servicing entity, and the servicing entity effecting review of the collected data so as to provide reimbursement to the caregiver.

21. The system of claim 20, further including:
a data processing entity, the data processing entity inputting the collected data from the data input portion, the data processing entity processing the collected data to generate processed collected data; and the data processing entity outputting the processed collected data to the servicing entity.

22. The system of claim 21, wherein the data processing entity outputs the processed collected data to the servicing entity using XML.

23. The method of claim 20, wherein the first data set consists of a caregiver code.

24. A method for documenting home care services for providing reimbursement in the situation where a claimant has a relationship with a servicing entity, the claimant being cared for by a caregiver, the method comprising:
a caregiver entering the home of a claimant;
the caregiver interfacing with a data input portion, which is disposed in the home of the claimant, prior to providing a service to the claimant so as to input a first data set into the data input portion;
the caregiver providing the service to the claimant;
upon completion of the service, the caregiver again interfacing with the data input portion to identify completion of the service, the first data set constituting collected data; and
transmitting the collected data to the servicing entity; and
wherein the first data set consists of a caregiver code;
wherein the transmitting the collected data to the servicing entity is performed using a data processing entity, and wherein:
the data processing entity inputting the collected data from the data input portion;
the data processing entity processing the collected data to generate processed collected data; and
the data processing entity outputting the processed collected data to the servicing entity; and
further including, after transmitting the collected data to the servicing entity, the servicing entity determining the service type and the hourly rate based on the caregiver code.

25. The method of claim 24 further including the servicing entity determining a reimbursement amount based on the collected data, the reimbursement amount being the amount of monies to be forwarded from the servicing entity to the claimant so that the claimant may be reimbursed for payments to the caregiver.
